(19) United States
(12) Patent Application Publication
(10) Pub. No.: US 2005/0214232 A1
(43) Pub. Date: Sep. 29, 2005

(54) Dental whitening

(75) Inventors: Alan B. Matthews, Santa Maria, CA (US); Kelly Smith-Reynolds, San Wisobispo, CA (US)

Correspondence Address: FISH & RICHARDSON P.C., P.O. BOX 1022, MINNEAPOLIS, MN, 55440-1022, US

(73) Assignee: The Gillette Company, a Delaware corporation

(21) Appl. No.: 11/098,771

(22) Filed: Apr. 4, 2005

Related U.S. Application Data
(63) Continuation of application No. 10/170,467, filed on Jun. 14, 2002, now abandoned.

(51) Int. Cl. A61K 7/20

(52) U.S. Cl. 424/53

(57) ABSTRACT

Teeth whitening is disclosed using a wand applicator that may apply a whitening composition directly to the one or more teeth to be whitened. The teeth whitening may include capping a whitening composition container with a wand applicator. Kits and whitening compositions for the same are also disclosed.

### TECHNICAL FIELD

The present invention relates generally to dental whitening.

### BACKGROUND ART

Currently, dental whitening, or bleaching, typically involves either application of a whitening gel by a dental professional in his/her office (a “chairside application”), application of a whitening gel using a tray or mouthguard that is either custom-made, preformed, or formed by the user prior to application of the gel (e.g., a “boil and bite”tray), or brushing with a dentifrice that provides a whitening agent sometimes with other hygienically-beneficial agents. These practices rely either on a costly professional application, use of a mouthguard or tray, or repeated use of a toothpaste containing whitening agents.

### SUMMARY

In one embodiment, the present invention provides a method for treating one or more teeth. The method includes the steps of applying a therapeutically effective amount of a whitening composition to one or more teeth using a wand applicator; leaving the whitening composition in contact with the one or more teeth such that the whitening composition may whiten the tooth; and capping a separate whitening composition container with said wand applicator.

In another embodiment, the invention provides a kit for treating one or more teeth. In such an embodiment the kit may include a wand applicator having a cap portion with a threaded configuration and an applicator portion extending from said cap portion; a whitening composition container having an interior space to hold a whitening composition and having a threaded configuration complementary to the threaded configuration of said wand applicator; and a whitening composition within said whitening composition container.

In another embodiment, the invention is a method for treating one or more teeth. The method includes the steps of using a wand applicator to apply a whitening composition to one or more teeth shortly after the one or more teeth are in contact with a material capable of staining the one or more teeth; leaving the whitening composition in contact with the one or more teeth such that the whitening composition may whiten the one or more teeth; and capping a separate whitening composition container with said wand applicator.

In another embodiment, the invention is a directed to a method for treating one or more teeth. This method includes the steps of providing a whitening composition container configured to hold a whitening composition and having a cap with a wand applicator connected thereto, said whitening composition container being substantially sealable by said cap; storing a whitening composition within a whitening composition container; and applying said whitening composition to one or more teeth.

Another embodiment of the invention involves a composition for whitening teeth. The composition may include one or more whitening agents present in an amount from about 3 to about 6 weight percent; one or more tackifying agents present in an amount from about 0.5 to about 3 weight percent; and one or more solubilizing agents present in an amount from about 1 to about 5 weight percent.

Another embodiment of the invention is directed to a composition having the following ingredients, listed by weight percent:

| INGREDIENT DESCRIPTION | % w/w |
|---|---|
| Water | 10-81 |
| Hydrogen Peroxide Solution | 3-6 |
| PEG 40 Hydrogenated Castor Oil | 1.7-4.3 |
| Triethanolamine | 0.6-5.4 |
| Carbomer | 0.5-3 |
| Flavor(s) | 0.6-2.5 |
| Sodium Saccharin | 0.06-0.35 |

### BRIEF DESCRIPTION OF THE DRAWINGS

Features of certain embodiments of the present invention will become apparent to those skilled in the art from the following description with reference to the figures, in which:

FIG. 1 depicts a cross-sectional view of an exemplary device for use in accordance with one embodiment of the invention;

FIG. 2 depicts another view of a device similar to that shown in FIG. 1 for use in accordance with certain embodiments of the invention; and

FIG. 3 depicts another view of a device for use with certain embodiments of the invention.

### DETAILED DESCRIPTION OF PREFERRED EMBODIMENTS

For simplicity and illustrative purposes, the principles of the present invention are described by referring to various exemplary embodiments thereof. Although the preferred embodiments of the invention are particularly disclosed herein, one of ordinary skill in the art will readily recognize that the same principles are equally applicable to, and can be implemented in other systems, and that any such variation would be within such modifications that do not part from the scope of the present invention. Before explaining the disclosed embodiments of the present invention in detail, it is to be understood that the invention is not limited in its application to the details of any particular arrangement shown, since the invention is capable of other embodiments. The terminology used herein is for the purpose of description and not of limitation. Further, although certain methods are described with reference to certain steps that are presented herein in certain order, in many instances, these steps may be performed in any order as would be appreciated by one skilled in the art, and the methods are not limited to the particular arrangement of steps disclosed herein.

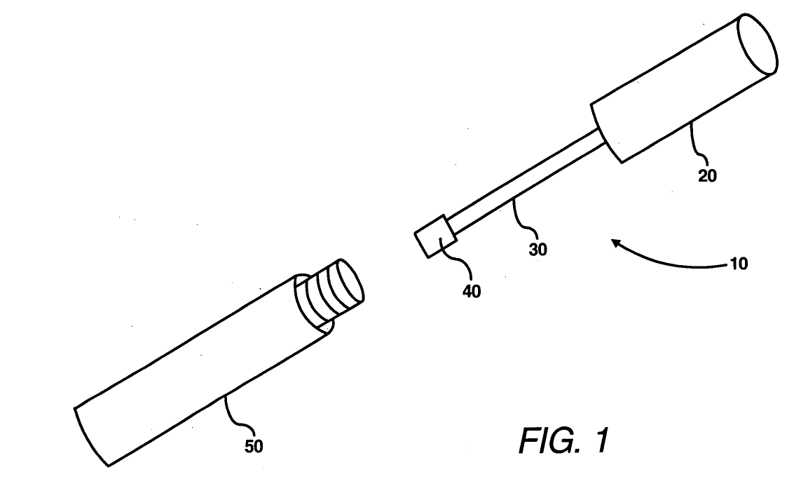

FIG. 1 depicts a wand applicator 10 for use in accordance with one embodiment of the invention. The wand applicator 10 may include a cap member 20 and an elongated stem member 30. The stem member 30 may have an application member 40 attached at one end of the stem member 30. The wand applicator 10 may be configured to be removably attached to a whitening composition container 50. This attachment may be through any suitable attachment means, such as screw-type threads on complementary portions of each of the wand applicator 10 and the whitening composition container 50. For instance, the cap member 20 may screw onto the whitening composition container 50, alternatively these components may engage through any suitable attachment means including, but not limited to, snapping together.

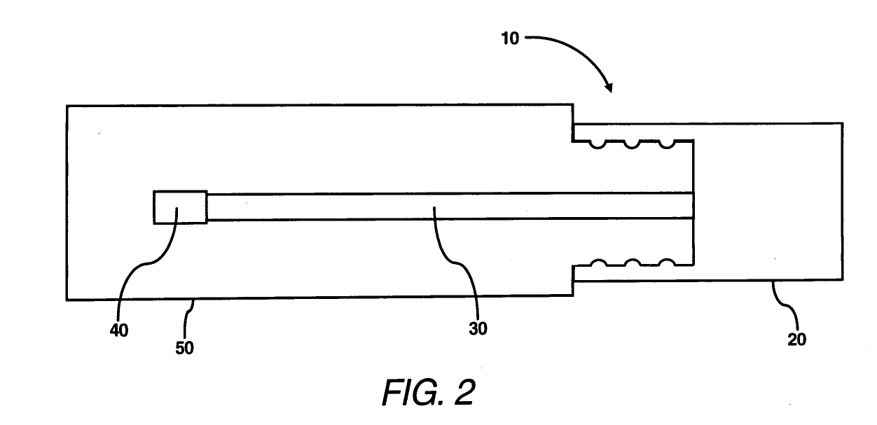

FIG. 2 depicts the wand applicator 10 threadedly engaged to the whitening composition container 50. From this figure, it can be appreciated that if the whitening composition container 50 held a fluid (not shown), the wand applicator 10 could be in contact with the fluid and some of the fluid could adhere to the wand applicator 10.

The following features and embodiments are described with reference to FIGS. 1 and 2. One embodiment includes a method. The method comprises the steps of applying a therapeutically effective amount of a whitening compound to a tooth using a wand applicator 10, leaving the whitening composition in contact with the tooth such that the whitening composition may whiten the tooth, and capping a separate whitening composition container 50 with the wand applicator 10. It can be appreciated that certain embodiments of the present invention simplify what was heretofore a cumbersome process. Certain embodiments of the present invention facilitate quick and relatively easy application of tooth whitening compositions and enable the easy treatment and maintenance of teeth.

In one embodiment, the invention involves the further step of applying the whitening composition to the wand applicator 10. This step may be achieved by bringing the wand applicator 10 into contact with the whitening composition, for instance by dipping the stem member 30 of the wand applicator 10 into the whitening composition.

In another embodiment, the invention includes repeating one or more of the steps of (i) applying a therapeutically effective amount of a whitening composition to one or more teeth, and/or (ii) applying said whitening composition to the wand applicator 10. Thus, it may be appreciated that certain embodiments of the invention include repeating various steps of a method of the invention. In another embodiment, the step of applying a therapeutically effective amount of a whitening composition to one or more teeth is performed periodically by a user. For instance, a user who is awake for sixteen hours a day might apply the whitening composition 5 times each day, at periodic intervals. Other users may apply the whitening composition more or less frequently, to suit their preferences. Some users may prefer to arrange to be awoken during the night in order to apply the whitening composition.

In yet another embodiment of the invention, the step of wiping the wand applicator 10 with a wiper (not shown) is included. This wiper may be connected to the whitening composition container 50 such that as the wand applicator 10 is withdrawn from the whitening composition container 50 excess whitening composition is removed from the wand applicator 10. The wiper may be constructed from any suitable material, and in a relatively simple embodiment, the wiper may simply be a flat surface or an edge provided near an opening on the whitening composition container 50.

The whitening composition may include a whitening agent and a tackifying agent, among other ingredients. Suitable whitening, or bleaching, agents include peroxides, such as hydrogen peroxide, carbamide peroxide or any known whitening compounds suitable for dental applications. Peroxide solutions are commercially available in varying strength, for instance H₂O₂ is available in strengths from 35 to 70%. The concentration in the composition would vary in accordance with the strength used. Therefore, the weight percent of the solution employed would be adjusted to account for the strength of solution used, as may be appreciated by one skilled in the art. In one embodiment, the whitening agent may be present in an amount from 3 to 6 weight percent. The whitening composition may be thin enough to be easily dispensed from the whitening composition container 50, yet have sufficient body and adhesion to remain on the dental surface following application, in order to accomplish whitening. Thus, the whitening composition need not remain on the dental surface indefinitely, however, it should be such that it is not quickly washed off by moisture normally present in the mouth.

A relatively sticky material may be included in the whitening composition to thicken the composition and act as a tackifying agent. This sticky material may be a polymer and may be selected from the group of carbomers known to resist degradation in the presence of peroxides and/or other whitening agents. Suitable carbomers are those that are safe for oral use and will not significantly inhibit whitening. Carbomers generally include acrylic acid backbones and small amounts of polyalkenyl polyether crosslinking agents. A suitable material is carboxypolymethylene. Carboxypolymethylene can be used to form a glue-like dental whitening composition that itself can act as an adhesive such that the composition tends to remain against a person's teeth. This tackifying agent may be present in an amount from about 0.5 to about 3 weight percent.

Carboxypolymethylene includes vinyl polymers having active carboxyl groups. Suitable carboxypolymethylene compositions may be obtained from B. F. Goodrich Company under the tradename CARBOPOL. Carboxypolymethylene is also generally known as carbomer. Different embodiments of the invention may utilize a variety of forms of salts of carboxypolymethylene including complete salts (where all of the acid groups have been neutralized) or partial salts (where only a portion of the acid groups have been neutralized). Further, certain embodiments of the invention may use mixtures of complete and partial salts.

The amount of carboxypolymethylene within the dental whitening compositions may vary with the identities and concentrations of other ingredients with the whitening composition, as well as with the desired level of tackiness of the overall composition. In order to increase the stickiness, viscosity, and resistance to dilution to saliva, one may adjust the concentration of carboxypolymethylene to achieve a desired level of any or all of these properties.

It should be understood that the sticky matrix material may include other tackifying agents that in combination with, or in lieu of some or all of, the carboxypolymethylene may yield a whitening composition for dental applications with appropriate levels of stickiness and other characteristics. Other synthetic polymers and/or natural gums, proteins, or other gel-forming mixtures can be used so long as they yield a relatively sticky whitening composition for dental applications.

In addition to the aforementioned whitening agents and tackifying agents, it may be desirable to include a flavor enhancing agent such as peppermint or spearmint oil. Further, a sweetener, such as saccharin, or sodium saccharin may be included in the composition. Any suitable flavor enhancing agent or a combination thereof may be included within the whitening composition. These flavoring agents may be present in amounts ranging from trace amounts 0.06 weight percent or less up to about 3 weight percent, or more as desired by the user.

Embodiments of the invention may include a variety of other ingredients. These ingredients may include a solvent, such as water, glycerin, propylene glycol, polyethylene glycol, or a variety of other compounds suitable for use as solvents for oral applications. The solvent may be present in an amount from about 10 to about 81 weight percent. The composition may also include a solubilizing agent, or emulsifying agent, such as ethoxylated castor oil. Similarly, any known solubilizing agent suitable for oral applications may be employed. Other suitable solubilizing agents include hydrogenated castor oil, or PEG 40 hydrogenated castor oil. The solubilizing agent may be present from about 1.5 to about 4.25 weight percent.

A neutralizing agent, such as a hydroxide, for instance, potassium hydroxide, sodium hydroxide, or ammonium hydroxide, may also be added to the composition. Another suitable neutralizing agent is triethanolamine. The neutralizing agent is such that helps to provide a suitable pH for the overall composition. The neutralizing agent may also serve to cross-link the thickening agent or tackifying agent to form a suitable gel. In one embodiment, the neutralizing agent is present from about 0.6 to about 5.4 weight percent. The suitable pH ranges from about 5.6 to about 7.6.

One embodiment of the invention may be a whitening composition suitable for application directly to the one or more teeth to be treated. A suitable whitening composition may have the following ingredients, listed by weight percent:

| INGREDIENT DESCRIPTION | % w/w |
|---|---|
| Water | 10-81 |
| Hydrogen Peroxide Solution | 3-6 |
| PEG 40 Hydrogenated Castor Oil | 1.7-4.3 |
| Triethanolamine | 0.6-5.4 |
| Carbomer | 0.5-3 |
| Flavor(s) | 0.6-2.5 |
| Sodium Saccharin | 0.06-0.35 |

Another suitable whitening composition may have the following ingredients, listed by weight percent:

| INGREDIENT DESCRIPTION | % w/w |
|---|---|
| Solvent(s) | 10-81 |
| Whitener(s) | 3-5.6 |
| Solubilizer(s) | 1.7-4.3 |
| Neutralizing Agent(s) | 0.6-6 |
| Tackifying Agent(s) | 0.5-3 |
| Flavor Agent(s) | 0.5-3 |

Yet another whitening composition may have the following ingredients, listed by weight percent:

| INGREDIENT DESCRIPTION | % w/w |
|---|---|
| Solvent(s) | 30-50 |
| Whitener(s) | 3-5 |
| Solubilizer(s) | 2-3 |
| Neutralizing Agent(s) | 2-6 |
| Tackifying Agent(s) | 1-3 |
| Flavor Agent(s) | 0.5-2 |

It should be appreciated that a suitable whitening composition may include one or more of any of the following: solvents, whiteners, solubilizers, neutralizing agents, tackifying agents, and flavor agents. In yet another embodiment, a nonaqueous formulation may be provided, for instance, with carbamide peroxide as a whitener.

In another embodiment, the invention is a kit for treating one or more teeth. The kit includes a wand applicator 10 having a cap portion 20 with a threaded configuration and applicator portion 30 extending from said cap portion 20. A whitening composition container 50 may also be provided having an interior space to hold a whitening composition and having a threaded configuration complementary to the threaded configuration of said wand applicator 10 of such that the whitening composition container 50 may connect to the wand applicator 10. The kit may also include a whitening composition within said whitening composition container 50. The whitening composition may be any suitable composition for whitening teeth and may include one or more whitening agents, thickening or tackifying agents, and/or flavoring agents, among other ingredients. This kit may include any of the compositions disclosed herein as whitening compositions.

The wand applicator 30 may have a tip member 40 attached at one end thereof. The tip member 40 serves to collect some of the whitening composition from the whitening composition container 50 such that the whitening composition may be applied to the surface of one or more teeth for treatment. The tip member 40 may be constructed from or may be any of a variety of suitable materials including, but not limited to, sponge-like or sponge material, rubber material, or a brush.

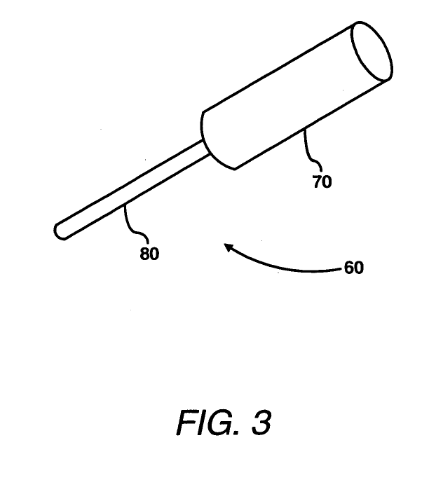

FIG. 3 shows another embodiment of an applicator wand 60. In this embodiment, the applicator portion 80 of the wand applicator 60 may be configured as a hollow tube having an edge defining an opening at the end of said applicator portion 80 and opposite said cap portion 70. In such an embodiment, capillary action may draw some of the whitening composition up into the hollow tube of the applicator portion 80, for later deposit upon the one or more teeth being treated. Thus, it may be appreciated that the wand applicator 60 may be any suitable device for serving as both a cap and an applicator, where the applicator may be used to withdraw the whitening composition from the whitening composition container 50 and to apply the whitening composition to the one or more teeth to be treated.

The following description is provided with reference to FIGS. 1 and 2. Another embodiment of the invention provides a method for treating one or more teeth. This method involves the steps of using a wand applicator 10 to apply a whitening composition to one or more teeth shortly after the one or more teeth are in contact with a material capable of staining the one or more teeth. Thus, a user may apply a whitening composition to one or more teeth, for instance, after eating and/or drinking food and beverage the user suspects might stain his or her teeth. In such an instance, earlier application of the whitening composition may achieve better results, though this may be limited by the user's activities and schedule.

A second step of this method involves leaving the whitening composition in contact with the one or more teeth such that the whitening composition may whiten the one or more teeth. The length of time that a given whitening composition adheres to a user's teeth will depend on not only the particular chemistry of the whitening composition but also the user's behavior. The whitening composition may generally be rubbed off, for instance, by any friction between the teeth and gums such as is created by talking or smiling. Thus, certain users might experience a decreased total adherence time for a given whitening composition, but generally a suitable whitening composition should adhere to the teeth being treated for a period ranging from between several minutes and extending up to several hours.

Another step of this method involves capping a separate whitening composition container 50 with the wand applicator 10. As can be appreciated from the foregoing description and accompanying drawings, the wand applicator 10 may serve as a cap to substantially seal the whitening composition container so that the entire assembly may be reused until all or substantially all of the whitening composition has been used. Thus, the method may also include the step of reapplying the whitening composition to one or more teeth.

In yet another embodiment of the invention, the whitening composition is applied to only a portion of one or more teeth. For instance, the portion of a single tooth may be stained or darkened, in which case the user might only need to apply a whitening composition to that portion that is so stained or darkened.

In another embodiment of the invention, the method is provided for treating one or more teeth including a step of providing a whitening composition container 50 configured to hold a whitening composition and having a cap portion 20 with an applicator portion 30 connected to the cap portion 20. The whitening composition container 50 may be substantially sealable by the cap portion 20. As previously described, this may be achieved a variety of ways, for instance the cap portion 20 may threadedly connect to the whitening composition container 50. A further step of this method may involve storing a whitening composition within the whitening composition container 50 and applying said whitening composition to one or more teeth.

Certain embodiments of the invention are described hereafter by the following examples, which are provided for descriptive purposes only and which are not intended to, and should not be read to, provide any limitations.

### EXAMPLE 1

| INGREDIENT DESCRIPTION | % w/w |
|---|---|
| Water | 80.080 |
| 50% Hydrogen Peroxide Solution | 8.000 |
| PEG 40 Hydrogenated Castor Oil | 2.500 |
| Triethanolamine | 5.020 |
| Carbomer | 2.800 |
| Flavor(s) | 1.400 |
| Sodium Saccharin | 0.200 |

The composition above is provided as an example and is not offered as the sole embodiment of the invention as one skilled in the art could be expected to achieve similar results with different compositions, including nonaqueous formulations containing other peroxides, for instance, carbamide peroxide.

### EXAMPLE 2

| INGREDIENT DESCRIPTION | % w/w |
|---|---|
| Water | 81.712 |
| 50% Hydrogen Peroxide Solution | 8.000 |
| PEG 40 Hydrogenated Castor Oil | 2.500 |
| Triethanolamine | 4.082 |
| Carbomer | 2.016 |
| Flavor(s) | 1.480 |
| Sodium Saccharin | 0.210 |

The whitening composition is prepared by first mixing the water and hydrogen peroxide solution, dissolving the sodium saccharin and dispersing the carbomer, then mixing until smooth. In a separate container, the flavor(s) are dispersed in the PEG 40 hydrogenated castor oil, then added to the aqueous phase, stirring until homogenous. Triethanolamine is then added to neutralize the carbomer and thicken the composition. The composition is then transferred to the containers, filling approximately ⅔rds to ⅘ths full. The wand applicator is then screwed on to the container. Users may be instructed on how to remove gel from the applicator and place a thin film on the surface of teeth to be whitened. Users may also be instructed to avoid eating or drinking for about 30 minutes for best results.

While the invention has been described with reference to certain exemplary embodiments thereof, those skilled in the art may make various modifications to the described embodiments of the invention without departing from the scope of the invention. The terms and descriptions used herein are set forth by way of illustration only and not meant as limitations. In particular, although the present invention has been described by way of examples, a variety of devices would practice the inventive concepts described herein. Although the invention has been described and disclosed in various terms and certain embodiments, the scope of the invention is not intended to be, nor should it be deemed to be, limited thereby and such other modifications or embodiments as may be suggested by the teachings herein are particularly reserved, especially as they fall within the breadth and scope of the claims here appended. Those skilled in the art will recognize that these and other variations are possible within the scope of the invention as defined in the following claims and their equivalents.

1. A method for treating one or more teeth comprising:
applying a therapeutically effective amount of a whitening composition including an ethoxylated castor oil to one or more teeth using a wand applicator wherein the whitening composition is contained in a container;
leaving the whitening composition in contact with the one or more teeth such that the whitening composition may whiten the tooth; and
capping said container with said wand applicator.

2. The method of claim 1, further comprising the step of applying said whitening composition to said wand applicator.

3. The method of claim 2, further comprising the step of repeating at least one of the steps of applying a therapeutically effective amount of a whitening composition to one or more teeth and applying said whitening composition to said wand applicator.

4. The method of claim 1, further comprising the step of removing a portion of said whitening composition from said whitening composition container using said wand applicator.

5. The method of claim 1, further comprising the step of wiping the wand applicator with a wiper attached to said.

6. The method of claim 1, wherein said step of applying a therapeutically effective amount of a whitening composition to one or more teeth using a wand applicator is performed periodically by a user.

7. The method of claim 1, wherein said whitening composition comprises the following, by weight percent:

| | |
|---|---|
| Water | 11.5-84; |
| Hydrogen Peroxide | 1.5-3; |
| Ethoxylated Castor Oil | 1.7-4.3; |
| Neutralizing Agent | 0.6-5.4; |
| Tackifying Agent | 0.5-3; |
| Flavor | 0.6-2.5; and |
| Sodium Saccharin | 0.06-0.35. |

8. The method of claim 1, wherein said whitening composition comprises: a whitening agent; and a tackifying agent.

9. The method of claim 8, wherein said whitening agent comprises a peroxide.

10. The method of claim 8, wherein said tackifying agent comprises a carbomer.

11. The method of claim 10, wherein said carbomer is substantially stable in the presence of one or more peroxides.

12. The method of claim 1, wherein said whitening composition comprises a peroxide.

13. The method of claim 12, wherein said peroxide comprises hydrogen peroxide.

14. The method of claim 1, wherein said ethoxylated castor oil comprises PEG 40 hydrogenated castor oil.

15. A kit for treating one or more teeth comprising:
a wand applicator having a cap portion with a threaded configuration and an applicator portion extending from said cap portion;
container having an interior space to hold a whitening composition and having a threaded configuration complementary to the threaded configuration of said wand applicator; and
a whitening composition including an ethoxylated castor oil within said whitening composition container.

16. The kit of claim 15, wherein said wand applicator further comprises: a tip member selected from the group consisting of: a sponge, a brush, or a rubber material connected to said applicator portion.

17. The kit of claim 15, wherein said applicator portion comprises: a hollow tube having an edge defining an opening at the end of said applicator portion and opposite said cap portion.

18. The kit of claim 15, wherein said whitening composition comprises: a whitening agent; and a tackifying agent.

19. The kit of claim 15, wherein said composition comprises a peroxide.

20. The kit of claim 18, wherein said tackifying agent comprises a carbomer.

21. The kit of claim 20, wherein said carbomer is substantially stable in the presence of one or more peroxides.

22-23. (canceled)

24. The kit of claim 15, wherein said ethoxylated castor oil comprises PEG 40 hydrogenated castor oil.

25. A method for treating one or more teeth comprising:
using a wand applicator to apply a whitening composition including an ethoxylated castor oil to one or more teeth shortly after the one or more teeth are in contact with a material capable of staining the one or more teeth, wherein the whitening composition is contained in a container;
leaving the whitening composition in contact with the one or more teeth such that the whitening composition may whiten the one or more teeth; and
capping the container with said wand applicator.

26. The method of claim 25, further comprising the step of: reapplying a whitening composition to one or more teeth.

27. The method of claim 25, wherein said step of using a wand applicator to apply a whitening composition to one or more teeth involves applying a whitening composition to a portion of one or more teeth.

28. A method for treating one or more teeth comprising:
providing a whitening composition container configured to hold a whitening composition and having a cap with a wand applicator connected thereto, said whitening composition container being substantially sealable by said cap;
storing a whitening composition including an ethoxylated castor oil within a whitening composition container; and
applying said whitening composition to one or more teeth.

29. A composition for whitening teeth comprising:
hydrogen peroxide;
one or more tackifying agents present in an amount from about 0.5 to about 3 weight percent; and
ethoxylated castor oil present in an amount from about 1 to about 5 weight percent.

30-31. (canceled)

32. The composition of claim 29, wherein said tackifying agent comprises one or more carbomers.

33. The composition of claim 29, wherein said tackifying agent comprises carboxypolymethylene.

34. (canceled)

35. The composition of claim 29, wherein said ethoxylated castor oil comprises PEG 40 hydrogenated castor oil.

36. A composition for whitening teeth having a pH of from about 5.6 to about 7.6 and comprising the following, by weight percent:

| | |
|---|---|
| Solvent | 11.5-84; |
| Hydrogen Peroxide | 1.5-3; |
| Ethoxylated Castor Oil | 1.7-4.3; |
| Neutralizing Agent | 0.6-5.4; |
| Tackifying Agent | 0.5-3; |
| Flavor | 0.6-2.5; and |
| Sodium Saccharin | 0.06-0.35. |

37. The method of claim 7, wherein the neutralizing agent is a hydroxide.

38. The method of claim 37, wherein the hydroxide is potassium hydroxide.

39. The method of claim 10, wherein the carbomer comprises carboxypolymethylene.

40. The composition of claim 29, wherein the composition comprises from about 0.5 to about 3 weight percent of the tackifying agent.
